SEQUENCE LISTING (27 sequences)

SEQ ID NO: 1 - DNA, 83 bp, Artificial Sequence
  source: /note=“Description of Artificial Sequence Synthetic oligonucleotide”
accagaatggccgagtggttaaggcgttggacttgggatccaatggattcatatccgcgtgggttcgaaccccacttctggta

SEQ ID NO: 2 - DNA, 83 bp, Artificial Sequence
  source: /note=“Description of Artificial Sequence Synthetic oligonucleotide”
accgggatggctgagtggttaaggcgttggacttgggatccaatggacaggtgtccgcgtgggttcgagccccactcccggta

SEQ ID NO: 3 - DNA, 72 bp, Artificial Sequence
  source: /note=“Description of Artificial Sequence Synthetic oligonucleotide”
ggctcgttggtctaggggtatgattctcgcttaaggtgcgagaggtcccgggttcaaatcccggacgagccc

SEQ ID NO: 4 - DNA, 72 bp, Artificial Sequence
  source: /note=“Description of Artificial Sequence Synthetic oligonucleotide”
ggctcgttggtctagtggtatgattctcgcttaaggtgcgagaggtcccgggttcaaatcccggacgagccc

SEQ ID NO: 5 - DNA, 83 bp, Artificial Sequence
  source: /note=“Description of Artificial Sequence Synthetic oligonucleotide”
gtcaggatggccgagtggtctaaggcgccagactagggttctggtctccaatggaggcgtgggttcgaatcccacttctgaca

SEQ ID NO: 6 - DNA, 84 bp, Artificial Sequence
  source: /note=“Description of Artificial Sequence Synthetic oligonucleotide”
gtcaggatggccgagtggtctaaggcgccagactagggttctggtctccgtatggaggcgtgggttcgaatcccacttctgaca

SEQ ID NO: 7 - DNA, 84 bp, Artificial Sequence
  source: /note=“Description of Artificial Sequence Synthetic oligonucleotide”
gtcaggatggccgagtggtctaaggcgccagactagggttctggtctccgcatggaggcgtgggttcgaatcccacttctgaca

SEQ ID NO: 8 - DNA, 83 bp, Artificial Sequence
  source: /note=“Description of Artificial Sequence Synthetic oligonucleotide”
accaggatggccgagtggttaaggcgttggactagggatccaatggacatatgtccgcgtgggttcgaaccccactcctggta

SEQ ID NO: 9 - DNA, 83 bp, Artificial Sequence
  source: /note=“Description of Artificial Sequence Synthetic oligonucleotide”
accgggatggccgagtggttaaggcgttggactagggatccaatgggctggtgcccgcgtgggttcgaaccccactctcggta

SEQ ID NO: 10 - DNA, 83 bp, Artificial Sequence
  source: /note=“Description of Artificial Sequence Synthetic oligonucleotide”
accagaatggccgagtggttaaggcgttggactagggatccaatggattcatatccgcgtgggttcgaaccccacttctggta

SEQ ID NO: 11 - DNA, 83 bp, Artificial Sequence
  source: /note=“Description of Artificial Sequence Synthetic oligonucleotide”
accaggatggccgagtggttaaggcgttggacttgggatccaatggacatatgtccgcgtgggttcgaaccccactcctggta

SEQ ID NO: 12 - DNA, 83 bp, Artificial Sequence
  source: /note=“Description of Artificial Sequence Synthetic oligonucleotide”
accgggatggccgagtggttaaggcgttggacttgggatccaatgggctggtgcccgcgtgggttcgaaccccactctcggta

SEQ ID NO: 13 - DNA, 83 bp, Artificial Sequence; identical to SEQ ID NO: 1
  source: /note=“Description of Artificial Sequence Synthetic oligonucleotide”

SEQ ID NO: 14 - DNA, 74 bp, Artificial Sequence
  source: /note=“Description of Artificial Sequence Synthetic oligonucleotide”
ggccggttagctcagttggttagagcgtggtgctaggaacgccaaggtcgcgggttcgatccccgtactggcca

SEQ ID NO: 15 - DNA, 74 bp, Artificial Sequence
  source: /note=“Description of Artificial Sequence Synthetic oligonucleotide”
ggccggttagctcagttggttagagcgtggtgctaggaacgccaaggtcgcgggttcgaaccccgtacgggcca

SEQ ID NO: 16 - DNA, 233 bp, Artificial Sequence
  source: /note=“Description of Artificial Sequence Synthetic polynucleotide”
ctcgagcagatacactgtctttgacacgttgatggattaagcgctccggtttttctgtgctgaacctcaggggacgccgacacacgtacacgtcgtcaggatggccgagtggtctaaggcgccagactagggttctggtctccaatggaggcgtgggttcgaatcccacttctgacagtcctttttttgtgctacggcgattcttggagagccagctgcgatcgctaggatcc

SEQ ID NO: 17 - DNA, 232 bp, Artificial Sequence
  source: /note=“Description of Artificial Sequence Synthetic polynucleotide”
ctcgagcagatacactgtctttgacacgttgatggattaagcgctccggtttttctgtgctgaacctcaggggacgccgacacacgtacacgtcgtcaggatggccgagtggtctaaggcgccagactagggttctggtctccaatggaggcgtgggttcgaatcccacttctgacagtcctttttttgtgctacggcgattcttggagagccagctgcgatcgctaggatc

SEQ ID NO: 18 - DNA, 234 bp, Artificial Sequence
  source: /note=“Description of Artificial Sequence Synthetic polynucleotide”
ctcgagcagatacactgtctttgacacgttgatggattaagcgctccggtttttctgtgctgaacctcaggggacgccgacacacgtacacgtcgtcaggatggccgagtggtctaaggcgccagactagggttctggtctccgtatggaggcgtgggttcgaatcccacttctgacagtcctttttttgtgctacggcgattcttggagagccagctgcgatcgctaggatcc

SEQ ID NO: 19 - DNA, 234 bp, Artificial Sequence
  source: /note=“Description of Artificial Sequence Synthetic polynucleotide”
ctcgagcagatacactgtctttgacacgttgatggattaagcgctccggtttttctgtgctgaacctcaggggacgccgacacacgtacacgtcgtcaggatggccgagtggtctaaggcgccagactagggttctggtctccgcatggaggcgtgggttcgaatcccacttctgacagtcctttttttgtgctacggcgattcttggagagccagctgcgatcgctaggatcc

SEQ ID NO: 20 - DNA, 233 bp, Artificial Sequence
  source: /note=“Description of Artificial Sequence Synthetic polynucleotide”
ctcgagcagatacactgtctttgacacgttgatggattaagcgctccggtttttctgtgctgaacctcaggggacgccgacacacgtacacgtcaccaggatggccgagtggttaaggcgttggactagggatccaatggacatatgtccgcgtgggttcgaaccccactcctggtagtcctttttttgtgctacggcgattcttggagagccagctgcgatcgctaggatcc

SEQ ID NO: 21 - DNA, 233 bp, Artificial Sequence
  source: /note=“Description of Artificial Sequence Synthetic polynucleotide”
ctcgagcagatacactgtctttgacacgttgatggattaagcgctccggtttttctgtgctgaacctcaggggacgccgacacacgtacacgtcaccgggatggccgagtggttaaggcgttggactagggatccaatgggctggtgcccgcgtgggttcgaaccccactctcggtagtcctttttttgtgctacggcgattcttggagagccagctgcgatcgctaggatcc

SEQ ID NO: 22 - DNA, 234 bp, Artificial Sequence
  source: /note=“Description of Artificial Sequence Synthetic polynucleotide”
ctcgagcagatacactgtctttgacacgttgatggattaagcgctccggtttttctgtgctgaacctcaggggacgccgacacacgtacacgtcgtcaggatggccgagtggtctaaggcgccagacttgggttctggtctccgcatggaggcgtgggttcgaatcccacttctgacagtcctttttttgtgctacggcgattcttggagagccagctgcgatcgctaggatcc

SEQ ID NO: 23 - DNA, 233 bp, Artificial Sequence
  source: /note=“Description of Artificial Sequence Synthetic polynucleotide”
ctcgagcagatacactgtctttgacacgttgatggattaagcgctccggtttttctgtgctgaacctcaggggacgccgacacacgtacacgtcaccaggatggccgagtggttaaggcgttggacttgggatccaatggacatatgtccgcgtgggttcgaaccccactcctggtagtcctttttttgtgctacggcgattcttggagagccagctgcgatcgctaggatcc

SEQ ID NO: 24 - DNA, 233 bp, Artificial Sequence
  source: /note=“Description of Artificial Sequence Synthetic polynucleotide”
ctcgagcagatacactgtctttgacacgttgatggattaagcgctccggtttttctgtgctgaacctcaggggacgccgacacacgtacacgtcaccgggatggccgagtggttaaggcgttggacttgggatccaatgggctggtgcccgcgtgggttcgaaccccactctcggtagtcctttttttgtgctacggcgattcttggagagccagctgcgatcgctaggatcc

SEQ ID NO: 25 - DNA, 224 bp, Artificial Sequence
  source: /note=“Description of Artificial Sequence Synthetic polynucleotide”
ctcgagcagatacactgtctttgacacgttgatggattaagcgctccggtttttctgtgctgaacctcaggggacgccgacacacgtacacgtcggccggttagctcagttggttagagcgtggcgctaagaacgccaaggtcgcgggttcgatccccgtacgggccagtcctttttttgtgctacggcgattcttggagagccagctgcgatcgctaggatcc

SEQ ID NO: 26 - DNA, 224 bp, Artificial Sequence
  source: /note=“Description of Artificial Sequence Synthetic polynucleotide”
ctcgagcagatacactgtctttgacacgttgatggattaagcgctccggtttttctgtgctgaacctcaggggacgccgacacacgtacacgtcggctggttagctcagttggttagagcgtggtgctaagaacgccaaggtcgcgggttcgatccccgtactggccagtcctttttttgtgctacggcgattcttggagagccagctgcgatcgctaggatcc

SEQ ID NO: 27 - DNA, 76 bp, Artificial Sequence
  source: /note=“Description of Artificial Sequence Synthetic oligonucleotide”
  source: /note=“Description of Combined DNA/RNA Molecule Synthetic oligonucleotide”
  modified_base (16)..(17): Dihydrouridine
  modified_base (39)..(39): Pseudouridine
  modified_base (55)..(55): Pseudouridine
gcggauuuagcucagnngggagagcgccagacugaayancuggagguccugugtncgauccacagaauucgcacca

(19) United States
(12) Patent Application Publication
(10) Pub. No.: US 2024/0318173 A1
(43) Pub. Date: Sep. 26, 2024

(54) SENSE,SUPPRESSOR TRANSFER RNA COMPOSITIONS AND RELATED USES AND FUNCTIONS

(71) Applicant: UNIVERSITY OF IOWA RESEARCH FOUNDATION, Iowa City, IA (US)

(72) Inventor: Christopher AHERN, Iowa City, IA (US)

(73) Assignee: UNIVERSITY OF IOWA RESEARCH FOUNDATION, Iowa City, IA (US)

(21) Appl. No.: 18/270,162

(22) PCT Filed: Dec. 29, 2021
(86) PCT No.: PCT/US2021/065508

§ 371 Date: Jun. 28, 2023

Related U.S. Application Data
(60) Provisional application No. 63/132,932, filed on Dec. 31, 2020.
(60) Provisional application No. 63/151,416, filed on Feb. 19, 2021.
(60) Provisional application No. 63/151,436, filed on Feb. 19, 2021.

(51) Int. Cl. C12N 15/113

(52) U.S. Cl. CPC C12N 15/113

(57) ABSTRACT

The present invention is related at least in part to sense, suppressor transfer RNAs (sstRNAs) comprising an acceptor stem and anticodon arm that comprises a noncognate triplet codon, as well as methods of use thereof.

### CROSS-REFERENCE TO RELATED APPLICATION

This application claims priority to U.S. Provisional Application No. 63/132,932 that was filed on Dec. 31, 2020, to U.S. Provisional Application No. 63/151,416 that was filed on Feb. 19, 2021, and to U.S. Provisional Application No. 63/151,436 that was filed on Feb. 19, 2021. The entire content of the applications referenced above is hereby incorporated by reference herein.

### BACKGROUND

DNA molecules carry genetic information in the form of the sequence of the nucleotide bases that make up the DNA polymer. Only four nucleotide bases are utilized in DNA: adenine, guanine, cytosine, and thymine. This information, in the form of codons of three contiguous bases is transcribed into messenger RNA (mRNA), and then translated by transfer RNA (tRNA) and ribosomes to form proteins. Four nucleotide bases are utilized in RNA: adenine, guanine, cytosine, and uracil. The genetic code is the relation between a triplet codon and a particular amino acid. Sixty-four possible codon triplets form the genetic code, where three stop (also called “terminating” or “nonsense”) codons provide a signal to the translation machinery (cellular ribosomes) to stop protein production at the particular codon. The other sixty-one codon triplets (also called “sense codons”) correspond to one of the 20 standard amino acids.

DNA is translated by ribosomes, causing each amino acid to be linked together one by one to form polypeptides, according to the genetic instructions specifically provided by the DNA. Transfer RNAs translate mRNA into a protein on a ribosome. Each tRNA contains an “anticodon” region that hybridizes with a complementary codon on the mRNA. A tRNA that carries its designated amino acid is called a “charged” tRNA. If the tRNA is one of the 61 amino acid-associated tRNAs (or “sense tRNAs”), it will normally attach its amino acid to the growing peptide. The structural gene of tRNA is about 72-90 nucleotides long and folds into a cloverleaf structure.

### SUMMARY OF THE INVENTION

It has been surprisingly found that tRNAs can be engineered such that they recognize a noncognate codon and carry an alternative amino acid for polypeptide production. These sense, suppressor tRNAs can be used to switch amino acids in polypeptides and proteins that can lead to protein expression or function disruption as well as decrease cell viability.

Thus, in one aspect a sense, suppressor transfer RNA (sstRNA) comprising an acceptor stem and an anticodon specific for a noncognate codon is provided.

In one embodiment of any one of the sstRNAs provided herein, the anticodon is specific for any noncognate codon. In one embodiment of any one of such sstRNAs provided herein, the acceptor stem is specific for any amino acid that is different from what is coded for by its anticodon.

In one embodiment of any one of the sstRNAs provided herein, the anticodon is specific for a leucine codon. In one embodiment of any one of such sstRNAs provided herein, the acceptor stem is specific for proline and can substitute leucine for proline in polypeptide or protein production. In one embodiment of any one of such sstRNAs provided herein, the sstRNA is encoded by a sequence as set forth in SEQ ID NOs: 3 and 4.

In one embodiment of any one of the sstRNAs provided herein, the anticodon is specific for a proline codon. In one embodiment of any one of such sstRNAs provided herein, the acceptor stem is specific for leucine and can substitute proline for leucine in polypeptide or protein production. In one embodiment of any one of such sstRNAs provided herein, the sstRNA is encoded by a sequence as set forth in SEQ ID NOs: 1, 2 and 5-13.

In one embodiment of any one of the sstRNAs provided herein, the anticodon is specific for a proline codon. In one embodiment of any one of such sstRNAs provided herein, the acceptor stem is specific for isoleucine and can substitute proline for isoleucine in polypeptide or protein production. In one embodiment of any one of such sstRNAs provided herein, the sstRNA is encoded by a sequence as set forth in SEQ ID NOs: 14 and 15.

In one embodiment of any one of the sstRNAs provided herein, the anticodon is specific for a codon with a missense mutation. In one embodiment of any one of such sstRNAs provided herein, the acceptor stem is specific for an alternative amino acid than what is coded for by the codon with the missense mutation and can substitute the alternative amino acid for polypeptide or protein production.

In another aspect, an oligonucleotide that encodes any one of the sstRNAs provided herein is provided. In one embodiment of any one of the oligonucleotides provided herein, the oligonucleotide has a total length of less than 150 or 300 nucleotides. In one embodiment of any one of the oligonucleotides provided herein, the oligonucleotide is DNA. In one embodiment of any one of the oligonucleotides provided herein, the oligonucleotide is RNA.

In another aspect, an expression cassette comprising a promoter and a nucleic acid encoding any one of the sstRNAs or any one of the oligonucleotides provided herein is provided. In one embodiment of any one of the expression cassettes provided herein, the expression cassette further comprises a terminator. In one embodiment of any one of the expression cassettes provided herein, the expression cassette comprises a nucleotide sequence as set forth in SEQ ID NOs: 16-26.

In another aspect, a vector comprising any one of the oligonucleotides or expression cassettes provided herein is provided. In one embodiment, the vector is a viral or plasmid vector.

In another aspect, any one of the oligonucleotides or expression cassettes provided herein are in the form of cDNA.

In another aspect, a composition comprising any one of the sstRNAs, any one of the oligonucleotides, any one of the expression cassettes, or any one of the vectors provided herein, and a pharmaceutically acceptable carrier is provided.

In another aspect, a composition comprising at least two or at least three of any one of the sstRNAs, at least two or at least three of any one of the oligonucleotides, at least two or at least three of any one of the expression cassettes, or at least two or at least three of any one of the vectors provided herein, and a pharmaceutically acceptable carrier is provided.

In one embodiment of any one of the compositions or methods provided herein, when there is at least two or three sstRNAs the at least two or three sstRNAs are in the same oligonucleotide, expression cassette, vector or composition. In another embodiment of any one of the compositions or methods provided herein, when there is at least two or three sstRNAs the at least two or three sstRNAs are in at least two or three different oligonucleotides, expression cassettes, vectors or compositions.

In another aspect, a cell comprising any one of the sstRNAs, any one of the oligonucleotides, any one of the expression cassettes or any one of the vectors provided herein is provided.

In another aspect, a cell comprising at least two or at least three of any one of the sstRNAs, at least two or at least three of any one of the oligonucleotides, at least two or at least three any one of the expression cassettes, or at least two or at least three of any one of the vectors provided herein is provided.

A method, comprising contacting cells with any one of the sstRNAs (e.g., at least one or at least two or at least three), any one of the oligonucleotides (e.g., at least one or at least two or at least three), any one of the expression cassettes (e.g., at least one or at least two or at least three), any one of the vectors (e.g., at least one or at least two or at least three), or any one of the compositions (e.g., at least one or at least two or at least three) provided herein is provided.

In another aspect, a method of modifying or disrupting protein expression or function, comprising delivering any one of the sstRNAs (e.g., at least one or at least two or at least three), any one of the oligonucleotides (e.g., at least one or at least two or at least three), any one of the expression cassettes (e.g., at least one or at least two or at least three), any one of the vectors (e.g., at least one or at least two or at least three), or any one of the compositions (e.g., at least one or at least two or at least three) provided herein is provided. In one embodiment of any one of such methods, the amount of the sstRNA(s), oligonucleotide(s), expression cassette(s), vector(s), or composition(s) is effective to modify or disrupt protein expression or function in the cells.

In another aspect, a method of killing cells, comprising contacting the cells with any one of the sstRNAs (e.g., at least one or at least two or at least three), any one of the oligonucleotides (e.g., at least one or at least two or at least three), any one of the expression cassettes (e.g., at least one or at least two or at least three), any one of the vectors (e.g., at least one or at least two or at least three), or any one of the compositions (e.g., at least one or at least two or at least three) provided herein is provided. In one embodiment of any one of such methods, the amount of the sstRNA(s), oligonucleotide(s), expression cassette(s), vector(s) or composition(s) is effective to kill cells. In one embodiment of any one of such methods, the cells are in vitro. In one embodiment of any one of such methods, the cells are in vivo.

In another aspect, a method of reducing cell survival, comprising contacting the cells with any one of the sstRNAs (e.g., at least one or at least two or at least three), any one of the oligonucleotides (e.g., at least one or at least two or at least three), any one of the expression cassettes (e.g., at least one or at least two or at least three), any one of the vectors (e.g., at least one or at least two or at least three), or any one of the compositions (e.g., at least one or at least two or at least three) provided herein is provided.

In another aspect, a method of reducing cell mobility, comprising contacting the cells with any one of the sstRNAs (e.g., at least one or at least two or at least three), any one of the oligonucleotides (e.g., at least one or at least two or at least three), any one of the expression cassettes (e.g., at least one or at least two or at least three), any one of the vectors (e.g., at least one or at least two or at least three), or any one of the compositions (e.g., at least one or at least two or at least three) provided herein is provided.

A method of activating immune cells, comprising contacting cells with any one of the sstRNAs (e.g., at least one or at least two or at least three), any one of the oligonucleotides (e.g., at least one or at least two or at least three), any one of the expression cassettes (e.g., at least one or at least two or at least three), any one of the vectors (e.g., at least one or at least two or at least three), or any one of the compositions (e.g., at least one or at least two or at least three) provided herein is provided. In one embodiment, the cells can come in contact with or be contacted with immune cells or other immune components such that an immune response is activated.

In another aspect, a method of treating a subject with a hyperproliferative disease or disorder, comprising administering to the subject any one of the sstRNAs (e.g., at least one or at least two or at least three), any one of the oligonucleotides (e.g., at least one or at least two or at least three), any one of the expression cassettes (e.g., at least one or at least two or at least three), any one of the vectors (e.g., at least one or at least two or at least three), or any one of the compositions (e.g., at least one or at least two or at least three) provided herein is provided. In one embodiment of any one of such methods, the amount of the sstRNA(s), oligonucleotide(s), expression cassette(s), vector(s) or composition(s) is effective to treat the hyperproliferative disease or disorder. In one embodiment of any one of such methods, the hyperproliferative disease or disorder is cancer. In one embodiment of any one of such methods, the cancer is melanoma or breast cancer, such as triple negative breast cancer.

In another aspect, a method of identifying an sstRNA is provided. In one embodiment of any one of the methods of identifying provided herein, the sstRNA is selected or engineered to comprise an anticodon specific for a noncognate codon. In one embodiment of any one of the methods of identifying provided herein, the sstRNA is one with an acceptor stem or arm specific for an amino acid different from the amino acid coded for by its anticodon. In one embodiment of any one of the methods of identifying provided herein, the method comprises or further comprises high-throughput cloning and screening. In one embodiment of any one of the methods of identifying provided herein, the method comprises or further comprises expressing the sstRNA in a cell, such as by transfection, and assessing protein expression or function modification or disruption and/or cell death, killing, survival or mobility. In one embodiment of any one of the methods of identifying provided herein, the method comprises or further comprises selecting sstRNA that result in protein expression or function modification or disruption and/or cell death, killing, survival or mobility.

### BRIEF DESCRIPTION OF THE DRAWINGS

FIG. 1 provides a table of the genetic code.

FIG. 2 shows the general four-arm structure of tRNAs comprising a T-arm, a D-arm, an anticodon arm, and an acceptor stem (or arm). These regions may also be referred to as ‘loops’ throughout.

FIG. 3 shows results from a co-transfection of SWTX2 and SWTX3 where GFP expression and cell division were rapidly mitigated.

FIG. 4 shows results from a co-transfection of SWTX5 and SWTX7.

FIG. 5 shows the plate map for a GFP plasmid library screen.

FIG. 6 shows results from plate/cell monitoring at 24 hours.

FIG. 7 shows results from plate/cell monitoring at 44 hours.

FIG. 8 shows the intensity of GFP at 44 hours in HEK293-T cells expressing individual plasmids.

FIG. 9 shows GFP expression 30 hours post-transfection in RPMI-7951 cells (a melanoma cell line) (GFP alone).

FIG. 10 shows GFP expression 30 hours post-transfection in RPMI-7951 cells (a melanoma cell line) (GFP+sstRNA 51).

FIG. 11 shows GFP expression results at 43 hours in HEK293T cells.

FIG. 12 shows GFP expression results at 43 hours in SKMEL3 cells (a melanoma cell line).

FIG. 13 shows results from a Fugene transfection with RPMI-7951 cells (P14) SWTX +/−tdtomato (at 1 μg/well) (2:1 Fugene:DNA ratio at 24 hrs).

FIG. 14 shows results from a Fugene transfection with RPMI-7951 cells (P14) SWTX +/−Nluc WT (at 1 μg/well) (2:1 Fugene:DNA ratio at 24 hrs).

FIG. 15 shows results from a Fugene transfection with RPMI-7951 cells (P14) SWTX +/−tdtomato (at 1 μg/well) (2:1 Fugene:DNA ratio at 48 hrs).

FIG. 16 shows results from a Fugene transfection with RPMI-7951 cells (P14) SWTX +/−Nluc WT (at 1 μg/well) (2:1 Fugene:DNA ratio at 48 hrs).

FIG. 17 shows results from a Lipofectamine 3000 transfection at 1:1.5, 1:3, and 1:6 ratios with cDNA of GFP, 51 or 51 plasmid. GFP signal is shown.

FIG. 18 shows results from Lipofetamine 3000 (LP) transfections, summary by ratio of reagent to cDNA in SKMEL melanoma line. Each plot shows two biological duplicates of the same condition.

FIG. 19 shows results from a HCC triple-negative breast cancer line.

FIG. 20 shows Fugene Transfected HEK293 cells for the indicated condition. A scratch assay was performed at 48 hours and imaged for GFP expression and cell density. Images at 72 hours show mobility/survival in GFP but not SWTX2+SWTX3 conditions.

FIG. 21 shows result with triple sstRNA combinations. The experimental conditions were as follows: gel was run at 150 V until at bottom; transfer occurred at 250 mA for 2 hr at 4° C.; block for 30 min @ RT in 5% milk in 1XTBST; primary O/N at 4° C. (1:2000 Sigma T2949 in blocking soln); wash with 1XTBST x3; secondary 1 hr @ RT in blocking soln (1:10000 anti-rabbit IgG); wash with 1XTBST x3; ECL imaging+“5 Shot”.

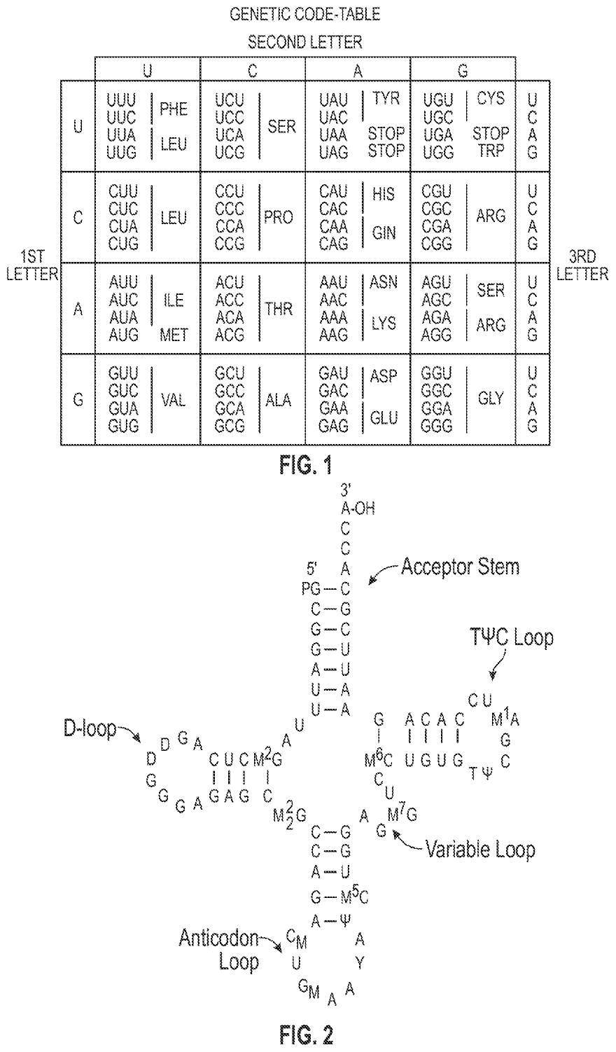

### DETAILED DESCRIPTION

Transfer RNA are decoders of DNA and RNA “blueprints” and help to make the proteins that form the structure of cells and tissues. These RNA molecules can be modified or engineered such that they can enable the systematic “recoding” of the genetic code. Provided herein is a platform technology based on site-directed changes in transfer RNA (tRNA) such that they can be used to deliver noncognate amino acids in polypeptide or protein production. For example, a tRNA, such as a proline tRNA, can be engineered to recognize and decode a noncognate codon, such as a leucine codon. In this example, the tRNA could be used to suppress the leucine codon and replace it with a proline during polypeptide or protein production. The biological impact of such targeted changes is that polypeptide or protein production can be adversely impacted, such as with modified, decreased expression and/or function. In the case of proline, as an example, as the amino acid promotes “kinks”, expressed polypeptides or proteins would generally have an altered shape and in turn disabled protein function. Other amino acid substitutions could also have adverse results. In some embodiments, the ultimate result may be cell death, killing, survival or mobility. Thus, the tRNAs provided herein can be used in the treatment of diseases or disorders where cell death, killing, survival or mobility would have a benefit. These sense, suppressor tRNAs can also be used to modify or disrupt protein expression and/or function, preferably the protein expression and/or function of an endogenous protein.

Thus, provided herein are sense, suppressor tRNAs that can suppress noncognate sense codons for the purpose of encoding alternative amino acids. As used herein, a “sense, suppressor transfer RNA (sstRNA)” refers to a tRNA that can carry, deliver or provide an amino acid different from the amino acid coded for by its anticodon. Preferably, the amino acid is a natural amino acid but different from the amino acid coded for by its anticodon. In other words, a sstRNA suppresses the amino acid coded for by its anticodon and substitutes a different amino acid in its place. As used herein, the amino acid coded for by its anticodon is the amino acid to which the anticodon is specific. As used herein, when referring to the amino acid specificity of the acceptor stem or arm, “specific for” refers to the amino acid that is or can be carried, delivered or provided by the acceptor stem or arm of the tRNA.

By altering anticodon loops of tRNA (the part that binds to RNA messages), novel tRNA sequences have been identified that possess the ability to switch protein codon meanings. For example, tRNA molecules have been generated that can decode proline codons as RNA into a structurally different amino acid, leucine; can decode leucine codons into proline; or can decode proline codons into isoleucine. In effect, switching the genetic meaning of the aforementioned codons. This technology has also been termed “SWTX” tRNA based on the acronym of Substitution with transfer RNA codon Exchange. The tRNAs provided herein are also referred to herein as sense, suppressor tRNAs. This therapeutic approach takes advantage of “code switching.” Code-switching by administering codon-selective amino acid conversion allows multiple routes to protein modification, such as changes to size, shape and/or charge. Any one or more of such changes can be a desired result in any one of the methods provided herein. Systematic and selective side-chain conversion can paralyze cellular function, halt cell division and/or growth, etc.

The nucleotide sequences encoding tRNAs can be generated synthetically. Also, nucleotide sequences encoding several hundred human tRNAs are known and generally available to those of skill in the art through sources such as Genbank. The structure of tRNAs is highly conserved and tRNAs can be functional across species. Thus, bacterial or other eukaryotic tRNA sequences are also potential sources for the tRNAs of the invention. The determination of whether a particular tRNA is functional as desired, such as in a desired mammalian cell, can be ascertained as described herein or through other experimentation that will be apparent to one of ordinary skill in the art with the benefit of the teachings provided herein.

The current studies show that a tRNA can be changed through molecular editing of the anticodon sequence within the tRNA. This approach allows for reprogramming a sense codon to be substituted with a noncognate amino acid. Engineered tRNAs as provided herein allow for “re-editing” of a codon to a specific amino acid. The small size of these tRNA molecules makes them amenable to ready expression, for example, a tRNA+the promoter is only ˜300 bp or less. A further advantage of the present invention is that it provides facile expression and cell delivery because the entire system can be compact. Briefly, an oligonucleotide can be synthesized that comprises the structural component of a tRNA gene functional in cells, such as in human cells. The sequence of this oligonucleotide is designed based upon the known sequence with substitutions made in the anticodon region of the tRNA causing the specific tRNA to recognize a particular codon but deliver an alternative amino acid.

tRNAs have a general four-arm structure comprising a T-arm, a D-arm, an anticodon arm, and an acceptor stem or arm (FIG. 2). The T-arm is made up of a “T-stem” and a “TY′C loop.” Any one of the tRNAs provided herein can comprise this four-arm structure. The tRNAs are approximately 100 nucleotides in length and can be readily introduced into cells.

In certain embodiments, the tRNA is encoded in an expression cassette. Because of the internal promoter sequences of tRNA encoding sequences, the tRNA sequence need not be included in a separate transcription unit, although one may be provided. Thus, the present invention also provides an expression cassette comprising a sequence encoding a tRNA as provided herein. In certain embodiments, the expression cassette further contains a promoter. In certain embodiments, the promoter is a regulatable promoter. In certain embodiments, the promoter is a constitutive promoter. The promoter to drive expression of the sequence encoding the tRNA to be delivered can be any desired promoter, selected by known considerations, such as the level of expression of a nucleic acid functionally linked to the promoter and the cell type in which the vector is to be used. Promoters can be an exogenous or an endogenous promoter.

“Expression cassette” as used herein means a nucleic acid sequence capable of directing expression of a particular nucleotide sequence in an appropriate cell, which may include a promoter operably linked to the nucleotide sequence of interest that may be operably linked to termination signals. The expression cassette including the nucleotide sequence of interest may be chimeric. The expression cassette may also be one that is naturally occurring but has been obtained in a recombinant form useful for heterologous expression. The expression of the nucleotide sequence in the expression cassette may be under the control of a constitutive promoter or of a regulatable promoter. The expression cassette may be or contained in a vector.

“Operably-linked” refers to the association of nucleic acid sequences on single nucleic acid fragment so that the function of one of the sequences is affected by another. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably linked to regulatory sequences in sense or antisense orientation.

The term “polypeptide” as used herein refers to a polymer of amino acids and includes full-length proteins and fragments thereof. Thus, “protein” and “polypeptide” are often used interchangeably herein.

The present method provides a method of delivering a nucleic acid to a cell. Administration to the cell can be accomplished by any means, including simply contacting the cell. The contact with the cells can be for any desired length of time. The cells can include any desired cell in humans as well as other large (non-rodent) mammals, such as primates, horse, sheep, goat, pig, and dog. Any one of the subjects provided herein can be a human or other mammal. The term “mammal” includes, but is not limited to, humans, mice, rats, guinea pigs, monkeys, dogs, cats, horses, cows, pigs, and sheep.

Suitable methods for the delivery and introduction into a subject are also provided or otherwise understood in the art. In one embodiment, pharmaceutical compositions will comprise sufficient genetic material to produce a therapeutically effective amount of the nucleic acid of interest, i.e., an amount sufficient to disrupt protein expression or function, to result in cell death, killing, survival or mobility, or to reduce or ameliorate symptoms of a disease state in question or an amount sufficient to confer a desired benefit.

The tRNAs can be delivered in an effective amount, and into a cell with tRNA synthetase, such as endogenous tRNA synthetase. A tRNA synthetase is considered to be “endogenous” to a cell if it is present in the cell into which a tRNA is introduced according to the present invention. As will be the apparent to those of ordinary skill in the art, a tRNA synthetase may be considered to be endogenous for these purposes whether it is naturally found in cells of the relevant type, or whether the particular cell at issue has been engineered or otherwise manipulated by the hand of man to contain or express it.

The pharmaceutical compositions will also contain a pharmaceutically acceptable excipient. Such excipients include any pharmaceutical agent that may be administered without undue toxicity. Pharmaceutically acceptable excipients include, but are not limited to, sorbitol, Tween80, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences (Mack Pub. Co., N.J. 1991).

As is apparent to those skilled in the art in view of the teachings of this specification, an effective amount of the tRNAs provided may be empirically determined. Administration can be effected in one dose, continuously or intermittently, throughout the course of treatment. Methods of determining the most effective means and dosages of administration may vary with the composition of the therapy, target cells, and the subject being treated, etc. Single and multiple administrations can be carried out with the dose level and pattern being selected by the treating physician.

Vehicles including water, aqueous saline, artificial CSF, or other known substances can be employed with the subject invention. To prepare a formulation, the purified composition can be isolated. The composition may then be adjusted to an appropriate concentration and packaged for use.

The term “nucleic acid” refers to deoxyribonucleotides or ribonucleotides and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides.

A “nucleic acid fragment” is a portion of a given nucleic acid molecule. The term “substantial identity” of polynucleotide sequences means that a polynucleotide comprises a sequence that has at least 70%, 71%, 72%, 73%, 74%, 75%, 76%, 77%, 78%, or 79%, or at least 80%, 81%, 82%, 83%, 84%, 85%, 86%, 87%, 88%, or 89%, or at least 90%, 91%, 92%, 93%, or 94%, or even at least 95%, 96%, 97%, 98%, or 99% sequence identity, compared to a reference sequence using one of the alignment programs described using standard parameters.

The exogenous genetic material (e.g., encoding a sstRNA or SWTX tRNA) can be introduced into a cell in vivo by genetic transfer methods, such as transfection. Various expression vectors (i.e., vehicles for facilitating delivery of exogenous genetic material into a target cell) are known to one of ordinary skill in the art. As used herein, “transfection of cells” refers to the acquisition by a cell of new genetic material by incorporation. Thus, transfection refers to the insertion of nucleic acid into a cell using physical or chemical methods. Several transfection techniques are known to those of ordinary skill in the art or are otherwise described herein.

As used herein, “exogenous genetic material” refers to a nucleic acid or an oligonucleotide, either natural or synthetic, that is not naturally found in the cells; or if it is naturally found in the cells, it is not transcribed or expressed at biologically significant levels by the cells. Thus, “exogenous genetic material” includes, for example, a non-naturally occurring nucleic acid that can be transcribed into a tRNA.

Typically, the exogenous genetic material can include the heterologous gene together with a promoter to control transcription of the new gene. The promoter characteristically has a specific nucleotide sequence necessary to initiate transcription. Optionally, the exogenous genetic material can further include additional sequences (i.e., enhancers) required to obtain the desired gene transcription activity. The exogenous genetic material may introduced into the cell genome immediately downstream from the promoter so that the promoter and coding sequence are operatively linked so as to permit transcription of the coding sequence.

In addition to at least one promoter and at least one heterologous nucleic acid, the expression vector may include a selection gene, for example, green fluorescent protein (GFP), for facilitating selection of cells that have been transfected with the expression vector. Alternatively, the cells can be transfected with two or more expression vectors, at least one vector containing the gene(s) encoding the tRNA, the other vector containing a selection gene. The selection of a suitable promoter, enhancer, selection gene and/or signal sequence (described below) is deemed to be within the scope of one of ordinary skill in the art without undue experimentation.

In an embodiment of any one of the embodiments provided herein, the sstRNA(s) are in the form of cDNA and can be delivered or contacted with cells as such.

In an embodiment of any one of the embodiments provided herein, the sstRNA(s) are charged or not charged with the desired amino acid and can be delivered or contacted with cells as such.

The present invention in one embodiment includes compositions and methods for any one of the methods or uses provided herein, such as for treating a hyperproliferative disease or disorder through administration of the sstRNAs or SWTX tRNAs of the invention. As used herein, “hyperproliferative disease or disorder” refers to any disease or disorder where there is an abnormally high rate of proliferation of cells by rapid division, substantial overproliferation, etc. Certain embodiments of the present disclosure provide a method of treating a hyperproliferative disease or disorder in a subject, such as a mammal. In certain embodiments, the mammal is human.

Certain embodiments of the present disclosure provide a use of a sstRNA, oligonucleotide, expression cassette, vector or composition as described herein to prepare a medicament useful for any one of the methods or uses provided herein, such as for treating a hyperproliferative disease or disorder in a subject, such as a mammal, such as a human. In certain embodiments, the therapy has potential use for the treatment/management of a hyperproliferative disease or disorder, including tumors, cancers, and neoplastic tissue, along with non-neoplastic or non-malignant hyperproliferative disorders. In certain embodiments, the hyperproliferative disease or disorder is cancer, such as melanoma or breast cancer, such as triple negative breast cancer. The cancer may also be lung cancer, colorectal cancer, prostate cancer, cervix/uterine cancer, bladder cancer or liver cancer.

The cancer may be one that is a resistant cancer. A “resistant cancer” is one that has been subjected to a treatment but has nevertheless progressed even with the treatment. In one embodiment the resistant cancer is a “pan-resistant cancer” where the cancer progresses despite more than one treatment, such as chemotherapy, radiation and/or targeted therapy.

The present disclosure also provides a cell containing a sstRNA, oligonucleotide, expression cassette, or vector described herein. The cell may be mammalian, such as human. According to one aspect, a cell expression system is provided. The expression system comprises a cell and an expression cassette as provided herein. Expression cassettes include, but are not limited to, plasmids, viral vectors, and other vehicles for delivering heterologous genetic material to cells.

The cell expression system can be formed in vivo. According to yet another aspect, a method for treating a subject in vivo is provided. The method includes introducing the sstRNA, oligonucleotide, expression cassette, ector, or composition to a subject in vivo. The subject may be mammalian, such as human.

The terms “treat” and “treatment” refer to both therapeutic treatment and measures that can alleviate symptoms or provide some benefit to a subject, wherein the object is to prevent or slow down (lessen) an undesired physiological change or disorder, such as the growth, development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition, disease or disorder.

The phrase “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, malignancies, etc. More particular examples of such cancers include melanoma or breast cancer, such as triple negative breast cancer.

The agents of the invention can be administered so as to result in a reduction in at least one symptom associated with a hyperproliferative disease or disorder (e.g., cancer). The amount administered will vary depending on various factors including, but not limited to, the composition chosen, the particular disease, the weight, the physical condition, and the age of the mammal. Such factors can be readily determined by the clinician employing animal models or other test systems that are known to the art.

Administration of the sstRNA, oligonucleotide, expression cassette, vector, or composition in accordance with the present invention may be continuous or intermittent, depending, for example, upon the recipient's physiological condition, whether the purpose of the administration is therapeutic, and other factors known to skilled practitioners. The administration of the agents of the invention may be essentially continuous over a preselected period of time or may be in a series of spaced doses.

One or more suitable unit dosage forms having the sstRNA, oligonucleotide, expression cassette, vector, or composition of the invention may be formulated and can be administered by a variety of routes. When the agents of the invention are prepared for administration, they may be combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof.

Pharmaceutical formulations containing the agents of the invention can be prepared by procedures known in the art using well-known and readily available ingredients. The agents of the invention can also be formulated as solutions appropriate for administration. The pharmaceutical formulations of the agents of the invention can also take the form of an aqueous or anhydrous solution or dispersion, or alternatively the form of an emulsion or suspension.

Thus, the agent may be formulated for administration and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in a suitable vehicle, e.g., sterile, pyrogen-free water, before use. It will be appreciated that the unit content of active ingredient or ingredients contained in an individual dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions such as phosphate buffered saline solutions pH 7.0-8.0 and water.

Any of the compositions provided herein can be placed in contact with, administered to or introduced into a cell with genetic transfer methods, such as transfection or transduction. Thus, any of the compositions provided herein can be included with or in a gene delivery vehicle. The gene delivery vehicle can be any delivery vehicle known in the art and can include naked nucleic acid that is facilitated by a receptor and/or lipid mediated transfection, as well as any of a number of vectors. Vectors include but are not limited to eukaryotic vectors, prokaryotic vectors (such as for example bacterial vectors) and viral vectors including, but not limited to, retroviral vectors, adenoviral vectors, adeno-associated viral vectors, lentivirus vectors (human and other including porcine), Herpes virus vectors, Epstein-Barr viral vectors, SV40 virus vectors, pox virus vectors, and pseudotyped viral vectors.

The term “retrovirus” is used in reference to RNA viruses that utilize reverse transcriptase during their replication cycle. There are several genera included within the family Retroviridae, including Cisternavirus A, Oncovirus A, Oncovirus B, Oncovirus C, Oncovirus D, Lentivirus, and Spumavirus. Retroviruses infect a wide variety of species and may be transmitted both horizontally and vertically.

As used herein, the term “lentivirus” refers to a group (or genus) of retroviruses that give rise to slowly developing disease. Viruses included within this group include HIV (human immunodeficiency virus; including HIV type 1, and HIV type 2), the etiologic agent of the human acquired immunodeficiency syndrome (AIDS); visna-maedi, that causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which cause immune deficiency and encephalopathy in sub-human primates. Diseases caused by these viruses are characterized by a long incubation period and protracted course. Usually, the viruses latently infect monocytes and macrophages, from which they spread to other cells. HIV, FIV, and SIV also readily infect T lymphocytes (i.e., T-cells).

In one embodiment, a viral vector is an AAV vector. An “AAV” vector refers to an adeno-associated virus and may be used to refer to the naturally occurring wild-type virus itself or derivatives thereof. The term covers all subtypes, serotypes and pseudotypes, and both naturally occurring and recombinant forms, except where required otherwise. As used herein, the term “serotype” refers to an AAV, which is identified by, and distinguished from other AAVs based on capsid protein reactivity with defined antisera, e.g., there are eight known serotypes of primate AAVs, AAV-1 to AAV-9 and AAVrh10. For example, serotype AAV2 is used to refer to an AAV, which contains capsid proteins encoded from the cap gene of AAV2 and a genome containing 5′ and 3′ ITR sequences from the same AAV2 serotype. As used herein, for example, rAAV1 may be used to refer an AAV having both capsid proteins and 5′-3′ ITRs from the same serotype or it may refer to an AAV having capsid proteins from one serotype and 5′-3′ ITRs from a different AAV serotype, e.g., capsid from AAV serotype 2 and ITRs from AAV serotype 5. For each example illustrated herein, the description of the vector design and production describes the serotype of the capsid and 5′-3′ ITR sequences. The abbreviation “rAAV” refers to recombinant adeno-associated virus, also referred to as a recombinant AAV vector (or “rAAV vector”).

An “AAV virus” or “AAV viral particle” refers to a viral particle composed of at least one AAV capsid protein (preferably by all of the capsid proteins of a wild-type AAV) and an encapsidated polynucleotide. If the particle comprises heterologous polynucleotide (i.e., a polynucleotide other than a wild-type AAV genome such as a transgene to be delivered to a mammalian cell), it is typically referred to as “rAAV”.

In one embodiment, the AAV expression vectors are constructed using known techniques to at least provide as operatively linked components in the direction of transcription, control elements including a transcriptional initiation region, the DNA of interest and a transcriptional termination region. The control elements are selected to be functional in a mammalian cell. The resulting construct which contains the operatively linked components is flanked (5′ and 3′) with functional AAV ITR sequences.

By “adeno-associated virus inverted terminal repeats” or “AAV ITRs” is meant the art-recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome.

Nucleotide sequences of AAV ITR regions are known. As used herein, an “AAV ITR” need not have the wild-type nucleotide sequence depicted, but may be altered, e.g., by the insertion, deletion or substitution of nucleotides. Additionally, the AAV ITR may be derived from any of several AAV serotypes, including without limitation, AAV1, AAV2, AAV3, AAV4, AAV5, AAV7, etc. Furthermore, 5′ and 3′ ITRs which flank a selected nucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell.

In one embodiment, AAV ITRs can be derived from any of several AAV serotypes, including without limitation, AAV1, AAV2, AAV3, AAV4, AAV5, AAV7, etc. Furthermore, 5′ and 3′ ITRs which flank a selected nucleotide sequence in an AAV expression vector need not necessarily be identical or derived from the same AAV serotype or isolate, so long as they function as intended, i.e., to allow for excision and rescue of the sequence of interest from a host cell genome or vector, and to allow integration of the DNA molecule into the recipient cell genome when AAV Rep gene products are present in the cell.

In one embodiment, AAV capsids can be derived from AAV2. Suitable DNA molecules for use in AAV vectors will be less than about 5 kilobases (kb), less than about 4.5 kb, less than about 4 kb, less than about 3.5 kb, less than about 3 kb, less than about 2.5 kb in size and are known in the art.

As used herein, the term “attenuated virus” refers to any virus (e.g., an attenuated lentivirus) that has been modified so that its pathogenicity in the intended subject is substantially reduced. The virus may be attenuated to the point it is nonpathogenic from a clinical standpoint, i.e., that subjects exposed to the virus do not exhibit a statistically significant increased level of pathology relative to control subjects.

As used herein, the term “packaging signal” or “packaging sequence” refers to sequences located within the viral genome or a vector that are required for, or at least facilitate, insertion of the viral or vector nucleic acid into the viral capsid or particle. The term “packaging signal” is also used for convenience to refer to a vector sequence that is transcribed into a functional packaging signal. A distinction between a packaging vector and a transgene vector can be that in the packaging vector, the major packaging signal is inactivated, and, in the transgene vector, the major packaging signal is functional. Ideally, in some embodiments, in the packaging vector, all packaging signals would be inactivated, and, in the transgene vector, all packaging signals would be functional. However, countervailing considerations, such as maximizing viral titer, or inhibiting homologous recombination, may lend such constructs less desirable.

The compositions provided herein can be contacted with cells or delivered or administered to a subject within a particle, such as a nanoparticle. A particle, such as a nanoparticle, can be, but is not limited to, lipid-based nanoparticles (also referred to herein as lipid nanoparticles, i.e., nanoparticles where the majority of the material that makes up their structure are lipids), virus-like particles (i.e., particles that are primarily made up of viral structural proteins but that are not infectious or have low infectivity), and/or particles with a combination of nanomaterials. The particles may be a variety of different shapes, including but not limited to spheroidal, cuboidal, pyramidal, oblong, cylindrical, toroidal, and the like.

In some embodiments, particles, such as nanoparticles, may comprise one or more lipids. In some embodiments, particles, such as nanoparticles, may comprise liposomes. In some embodiments, particles, such as nanoparticles, may comprise a lipid bilayer. In some embodiments, particles, such as nanoparticles, may comprise a lipid monolayer. In some embodiments, particles, such as nanoparticles, may comprise a micelle.

The invention is now illustrated by the following non-limiting Examples.

### EXAMPLES

### Example 1

Transfer RNA molecules have been generated where the anticodon loop has been modified so that it is specific for a noncognate triplet DNA codon. These SWTX (Substitution with fRNA exchange) tRNAs can suppress sense codons within the human genetic code (also referred to herein as sense, suppressor tRNAs (sstRNAs)).

Briefly, a single-plasmid design was used to express codon-swapped tRNA and GFP reporter. The tRNA cassette contains an upstream leader sequence followed by a downstream terminator. HEK 293 cells were reverse transfected as follows. Cells were passaged in culture daily for at least three days after thawing and grown to 70% confluency. Plasmid cDNA was mixed with transfection reagent and added to a 96-well, followed by HEK293 cells at 10K/180 μl density. Cells were then imaged for GFP expression at 24 and 48 hour time points.

### Results

SWTX tRNAs or sstRNAs have been generated that can deliver alternative amino acids. For example, SWTX2 and SWTS3 decode proline triplet codons to place a leucine at a proline (CCA) codon (lower case letters indicate anticodon triplet). In other words, a leucine tRNA was recoded to encode leucine at proline (CCA) codons and generated.

SWTX2:
(SEQ ID NO: 1)
ACCAGAATGGCCGAGTGGTtAAGGCGTTGGACTtggGATCCAATG
GATTCATATCCGCGTGGGTTCGAACCCCACTTCTGGTA
SWTX3:
(SEQ ID NO: 2)
ACCGGGATGGCTGAGTGGTtAAGGCGTTGGACTtggGATCCAATG
GACAGGTGTCCGCGTGGGTTCGAGCCCCACTCCCGGTA

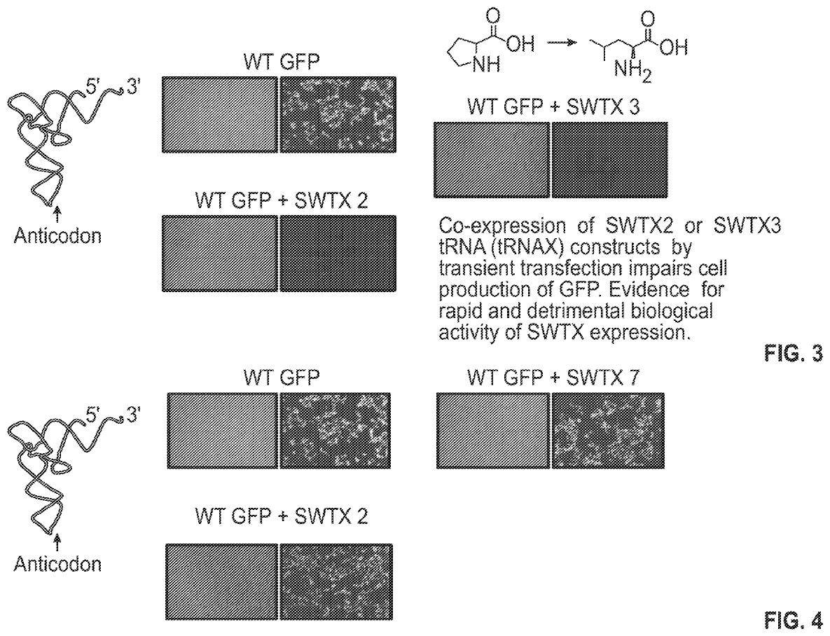

Co-transfection of these constructs rapidly mitigated GFP expression and cell division (FIG. 3). Co-expression of SWTX2 or SWTX3 tRNA constructs by transient transfection impaired cell production of GFP demonstrating rapid and detrimental biological activity as a result. Co-expression of SWTX5 or SWTX7 tRNA constructs results are also shown (FIG. 4).

### Example 2

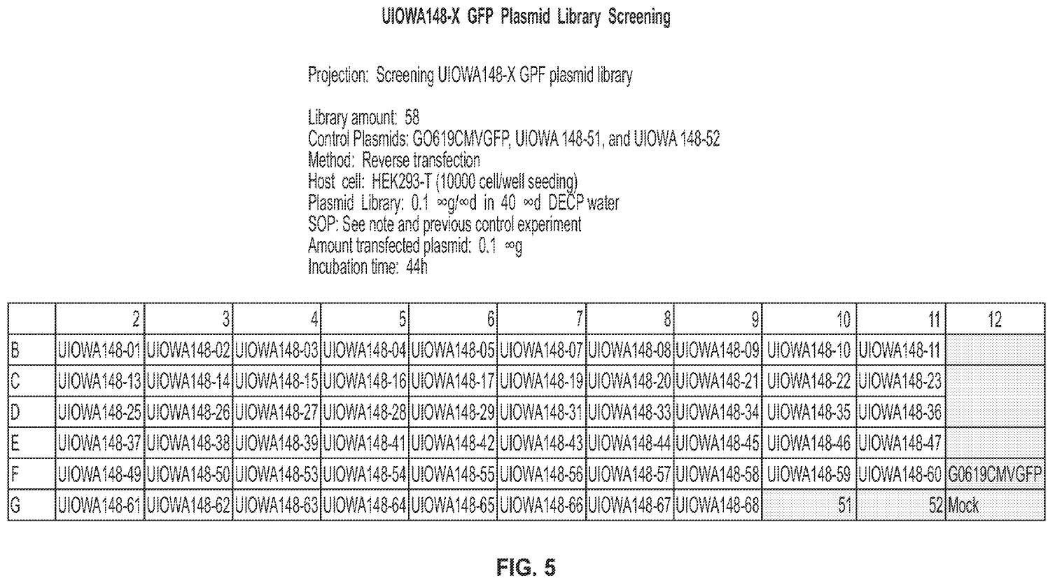

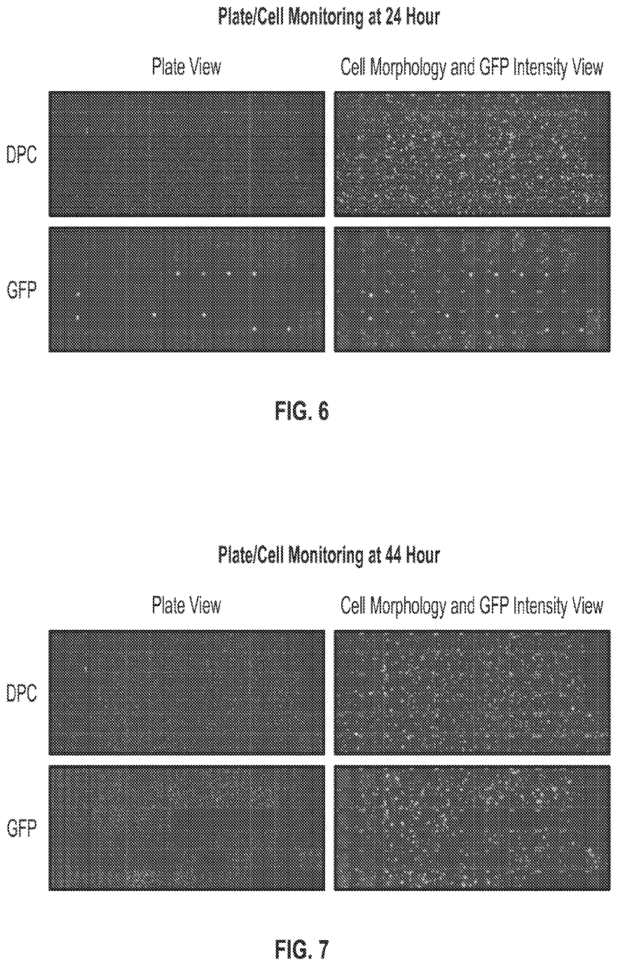

Further examples of tRNA constructs that were generated as described above, and for some screened when expressed in plasmids with respect to resulting GFP intensity (SWTX51 and 52 have an all-in-one plasmid design) (FIGS. 5-8), are as follows:

Pro -> Leu AAG (Suppress Leu and convert to Pro)
SWTX 11:
(SEQ ID NO: 3)
GGCTCGTTGGTCTAGGGGTATGATTCTCGCTTaagGTGCGAGAGG
tCCCGGGTTCAAATCCCGGACGAGCCC
SWTX 13:
(SEQ ID NO: 4)
GGCTCGTTGGTCTAGTGGTATGATTCTCGCTTaagGTGCGAGAGG
tCCCGGGTTCAAATCCCGGACGAGCCC
Leu -> Pro AGG (Suppress Pro and convert to Leu)
SWTX 31:
(SEQ ID NO: 5)
GTCAGGATGGCCGAGTGGTctAAGGCGCCAGACTaggGTTCTGGT
CTCCAATGGAGGCGTGGGTTCGAATCCCACTTCTGACA
SWTX 32:
(SEQ ID NO: 6)
GTCAGGATGGCCGAGTGGTctAAGGCGCCAGACTaggGTTCTGGT
CTCCGTATGGAGGCGTGGGTTCGAATCCCACTTCTGACA
SWTX 33:
(SEQ ID NO: 7)
GTCAGGATGGCCGAGTGGTctAAGGCGCCAGACTaggGTTCTGGT
CTCCGCATGGAGGCGTGGGTTCGAATCCCACTTCTGACA
SWTX 34:
(SEQ ID NO: 8)
ACCAGGATGGCCGAGTGGTtAAGGCGTTGGACTaggGATCCAATG
GACATATGTCCGCGTGGGTTCGAACCCCACTCCTGGTA
SWTX 35:
(SEQ ID NO: 9)
ACCGGGATGGCCGAGTGGTAAGGCGTTGGACTaggGATCCAATGG
GCTGGTGCCCGCGTGGGTTCGAACCCCACTCTCGGTA
SWTX 36:
(SEQ ID NO: 10)
ACCAGAATGGCCGAGTGGTtAAGGCGTTGGACTaggGATCCAATG
GATTCATATCCGCGTGGGTTCGAACCCCACTTCTGGTA
Leu -> Pro TGG (Suppress Pro and convert to Leu)
SWTX 50:
(SEQ ID NO: 11)
ACCAGGATGGCCGAGTGGTtAAGGCGTTGGACTtggGATCCAATG
GACATATGTCCGCGTGGGTTCGAACCCCACTCCTGGTA
SWTX 51:
(SEQ ID NO: 12)
ACCGGGATGGCCGAGTGGTtAAGGCGTTGGACTtggGATCCAATG
GGCTGGTGCCCGCGTGGGTTCGAACCCCACTCTCGGTA
SWTX 52:
(SEQ ID NO: 13)
ACCAGAATGGCCGAGTGGTtAAGGCGTTGGACTtggGATCCAATG
GATTCATATCCGCGTGGGTTCGAACCCCACTTCTGGTA
Ile -> Pro AGG (Suppress Pro and convert to Ile)
SWTX 55:
(SEQ ID NO: 14)
GGCCGGTTAGCTCAGTTGGTtAGAGCGTGGTGCTaggAACGCCAA
GGtCGCGGGTTCGATCCCCGTACTGGCCA
SWTX 57:
(SEQ ID NO: 15)
GGCCGGTTAGCTCAGTTGGTtAGAGCGTGGTGCTaggAACGCCAA
GGtCGCGGGTTCGAACCCCGTACGGGCCA

The plasmid sequences used are as follows:

SWTX 31
G0619 pFBAAVmcsCMVeGFPSV40pA
(SEQ ID NO: 16)
CTCGAGCAGATACACTGTCTTTGACACGTTGATGGATTAAGCGCT
CCGGTTTTTCTGTGCTGAACCTCAGGGGACGCCGACACACGTACA
CGTCGTCAGGATGGCCGAGTGGTctAAGGCGCCAGACTaggGTTC
TGGTCTCCAATGGAGGCGTGGGTTCGAATCCCACTTCTGACAGTC
CTTTTTTTGTGCTACGGCGATTCTTGGAGAGCCAGCTGCGATCGC
TAGGATCC
SWTX 32
G0619 pFBAAVmcsCMVeGFPSV40pA
(SEQ ID NO: 17)
CTCGAGCAGATACACTGTCTTTGACACGTTGATGGATTAAGCGCT
CCGGTTTTTCTGTGCTGAACCTCAGGGGACGCCGACACACGTACA
CGTCGTCAGGATGGCCGAGTGGTctAAGGCGCCAGACTaggGTTC
TGGTCTCCAATGGAGGCGTGGGTTCGAATCCCACTTCTGACAGTC
CTTTTTTTGTGCTACGGCGATTCTTGGAGAGCCAGCTGCGATCGC
TAGGATC
SWTX 33
G0619 pFBAAVmcsCMVeGFPSV40pA
(SEQ ID NO: 18)
CTCGAGCAGATACACTGTCTTTGACACGTTGATGGATTAAGCGCT
CCGGTTTTTCTGTGCTGAACCTCAGGGGACGCCGACACACGTACA
CGTCGTCAGGATGGCCGAGTGGTctAAGGCGCCAGACTaggGTTC
TGGTCTCCGTATGGAGGCGTGGGTTCGAATCCCACTTCTGACAGT
CCTTTTTTTGTGCTACGGCGATTCTTGGAGAGCCAGCTGCGATCG
CTAGGATCC
SWTX 34
G0619 pFBAAVmcsCMVeGFPSV40pA
(SEQ ID NO: 19)
CTCGAGCAGATACACTGTCTTTGACACGTTGATGGATTAAGCGCT
CCGGTTTTTCTGTGCTGAACCTCAGGGGACGCCGACACACGTACA
CGTCGTCAGGATGGCCGAGTGGTctAAGGCGCCAGACTaggGTTC
TGGTCTCCGCATGGAGGCGTGGGTTCGAATCCCACTTCTGACAGT
CCTTTTTTTGTGCTACGGCGATTCTTGGAGAGCCAGCTGCGATCG
CTAGGATCC
SWTX 35
G0619 pFBAAVmcsCMVeGFPSV40pA
(SEQ ID NO: 20)
CTCGAGCAGATACACTGTCTTTGACACGTTGATGGATTAAGCGCT
CCGGTTTTTCTGTGCTGAACCTCAGGGGACGCCGACACACGTACA
CGTCACCAGGATGGCCGAGTGGTtAAGGCGTTGGACTaggGATCC
AATGGACATATGTCCGCGTGGGTTCGAACCCCACTCCTGGTAGTC
CTTTTTTTGTGCTACGGCGATTCTTGGAGAGCCAGCTGCGATCGC
TAGGATCC
SWTX 36
G0619 pFBAAVmcsCMVeGFPSV40pA
(SEQ ID NO: 21)
CTCGAGCAGATACACTGTCTTTGACACGTTGATGGATTAAGCGCT
CCGGTTTTTCTGTGCTGAACCTCAGGGGACGCCGACACACGTACA
CGTCACCGGGATGGCCGAGTGGTtAAGGCGTTGGACTaggGATCC
AATGGGCTGGTGCCCGCGTGGGTTCGAACCCCACTCTCGGTAGTC
CTTTTTTTGTGCTACGGCGATTCTTGGAGAGCCAGCTGCGATCGC
TAGGATCC
SWTX 50
G0619 pFBAAVmcsCMVeGFPSV40pA
(SEQ ID NO: 22)
CTCGAGCAGATACACTGTCTTTGACACGTTGATGGATTAAGCGCT
CCGGTTTTTCTGTGCTGAACCTCAGGGGACGCCGACACACGTACA
CGTCGTCAGGATGGCCGAGTGGTctAAGGCGCCAGACTtggGTTC
TGGTCTCCGCATGGAGGCGTGGGTTCGAATCCCACTTCTGACAGT
CCTTTTTTTGTGCTACGGCGATTCTTGGAGAGCCAGCTGCGATCG
CTAGGATCC
SWTX 51
G0619 pFBAAVmcsCMVeGFPSV40pA
(SEQ ID NO: 23)
CTCGAGCAGATACACTGTCTTTGACACGTTGATGGATTAAGCGCT
CCGGTTTTTCTGTGCTGAACCTCAGGGGACGCCGACACACGTACA
CGTCACCAGGATGGCCGAGTGGTtAAGGCGTTGGACTtggGATCC
AATGGACATATGTCCGCGTGGGTTCGAACCCCACTCCTGGTAGTC
CTTTTTTTGTGCTACGGCGATTCTTGGAGAGCCAGCTGCGATCGC
TAGGATCC
SWTX 52
G0619 pFBAAVmcsCMVeGFPSV40pA
(SEQ ID NO: 24)
CTCGAGCAGATACACTGTCTTTGACACGTTGATGGATTAAGCGCT
CCGGTTTTTCTGTGCTGAACCTCAGGGGACGCCGACACACGTACA
CGTCACCGGGATGGCCGAGTGGTtAAGGCGTTGGACTtggGATCC
AATGGGCTGGTGCCCGCGTGGGTTCGAACCCCACTCTCGGTAGTC
CTTTTTTTGTGCTACGGCGATTCTTGGAGAGCCAGCTGCGATCGC
TAGGATCC
SWTX 55
G0619 pFBAAVmcsCMVeGFPSV40pA
(SEQ ID NO: 25)
CTCGAGCAGATACACTGTCTTTGACACGTTGATGGATTAAGCGCT
CCGGTTTTTCTGTGCTGAACCTCAGGGGACGCCGACACACGTACA
CGTCGGCCGGTTAGCTCAGTTGGTtAGAGCGTGGCGCTaagAACG
CCAAGGtCGCGGGTTCGATCCCCGTACGGGCCAGTCCTTTTTTTG
TGCTACGGCGATTCTTGGAGAGCCAGCTGCGATCGCTAGGATCC
SWTX 57
G0619 pFBAAVmcsCMVeGFPSV40pA
(SEQ ID NO: 26)
CTCGAGCAGATACACTGTCTTTGACACGTTGATGGATTAAGCGCT
CCGGTTTTTCTGTGCTGAACCTCAGGGGACGCCGACACACGTACA
CGTCGGCTGGTTAGCTCAGTTGGTtAGAGCGTGGTGCTaagAACG
CCAAGGtCGCGGGTTCGATCCCCGTACTGGCCAGTCCTTTTTTTG
TGCTACGGCGATTCTTGGAGAGCCAGCTGCGATCGCTAGGATCC

### Example 3

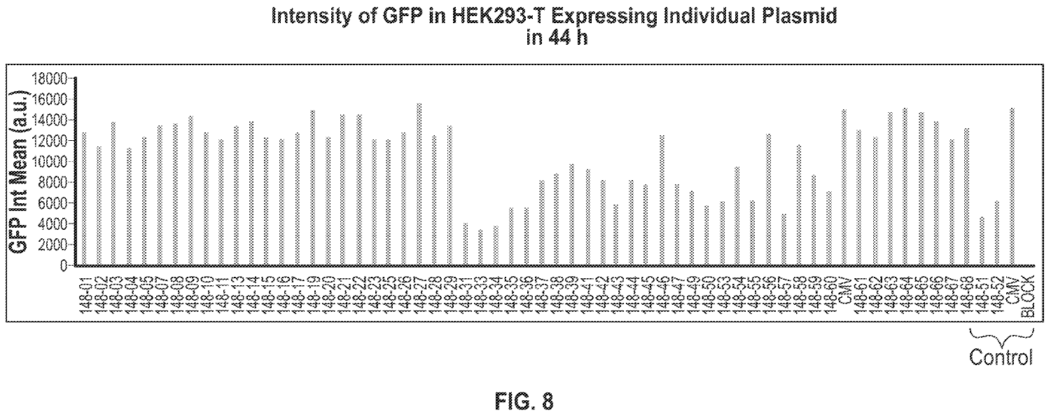

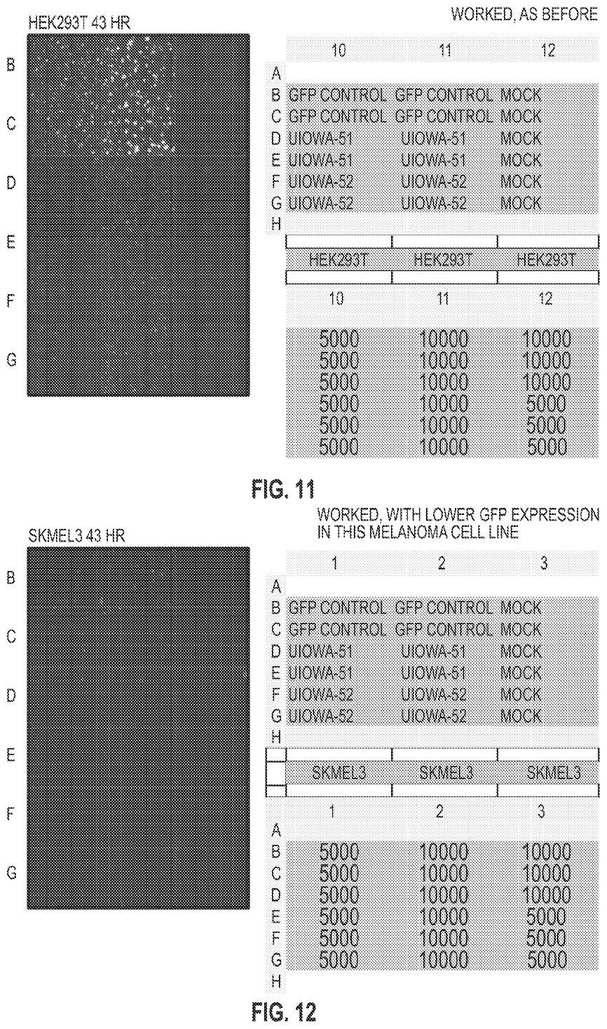

A number of the generated sstRNAs were also transfected (WT GFP or GFP+the SWTX 51 or SWTX 52 constructs, SWTX51 and 52 have an all-in-one plasmid design) in the following cell lines (HEK293T similar to above, FIGS. 8 and 11).

SK-MEL-3: Malignant melanoma, skin; derived from Metastatic Site: lymph node. The base medium for this cell line was ATCC-formulated McCoy's 5a Medium Modified, Catalog No. 30-2007. To make the complete growth medium, the following components were added to the base medium: fetal bovine serum to a final concentration of 15%. Reverse transfection was performed.

RPMI-7951: Malignant melanoma, skin; derived from metastatic site: lymph node. The base medium for this cell line was ATCC-formulated Eagle's Minimum Essential Medium (MEM), Catalog No. 30-2003. To make the complete growth medium, the following components were added to the base medium: fetal bovine serum to a final concentration of 10%. FUGENE was used as the transfection reagent.

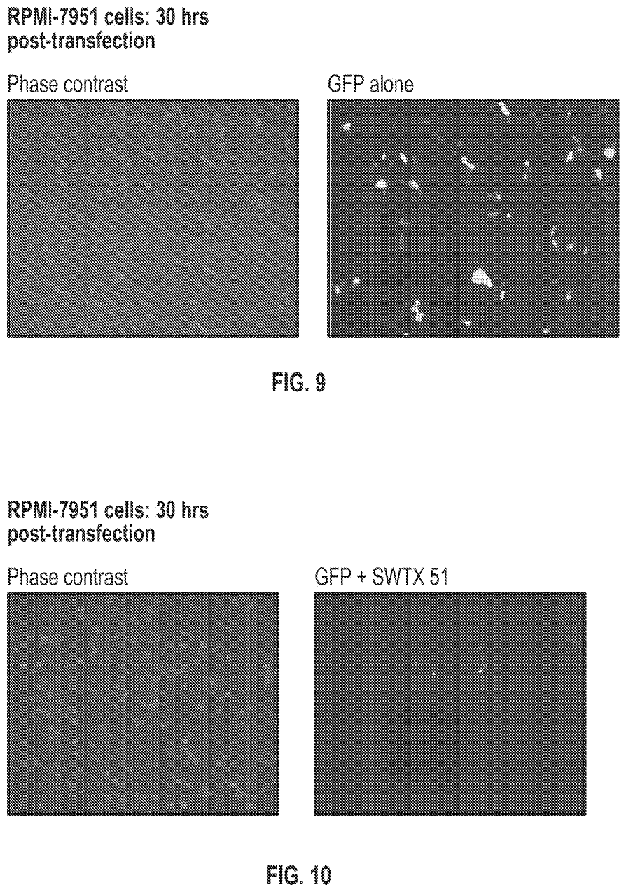

The results show that SWTX 51 and SWTX 52 impair protein production in the melanoma lines (FIGS. 9, 10 and 12).

### Example 4

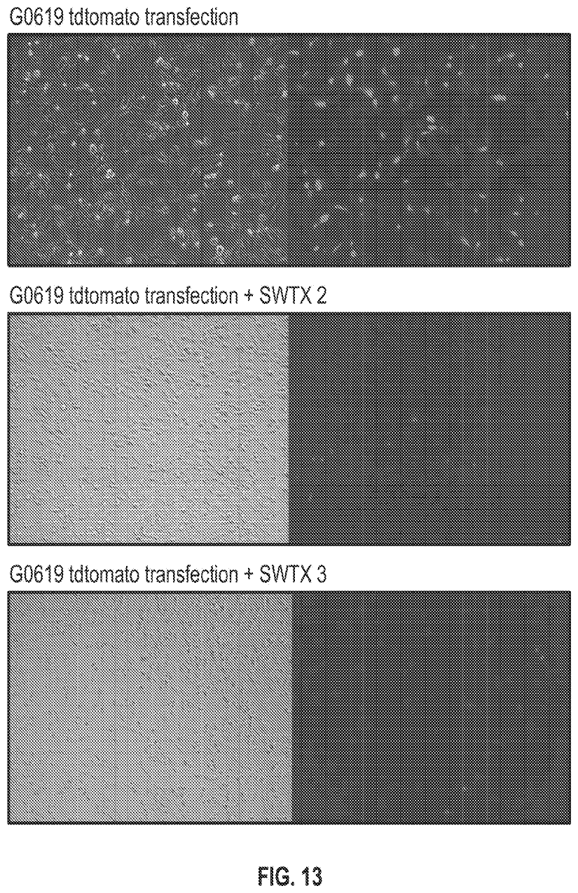

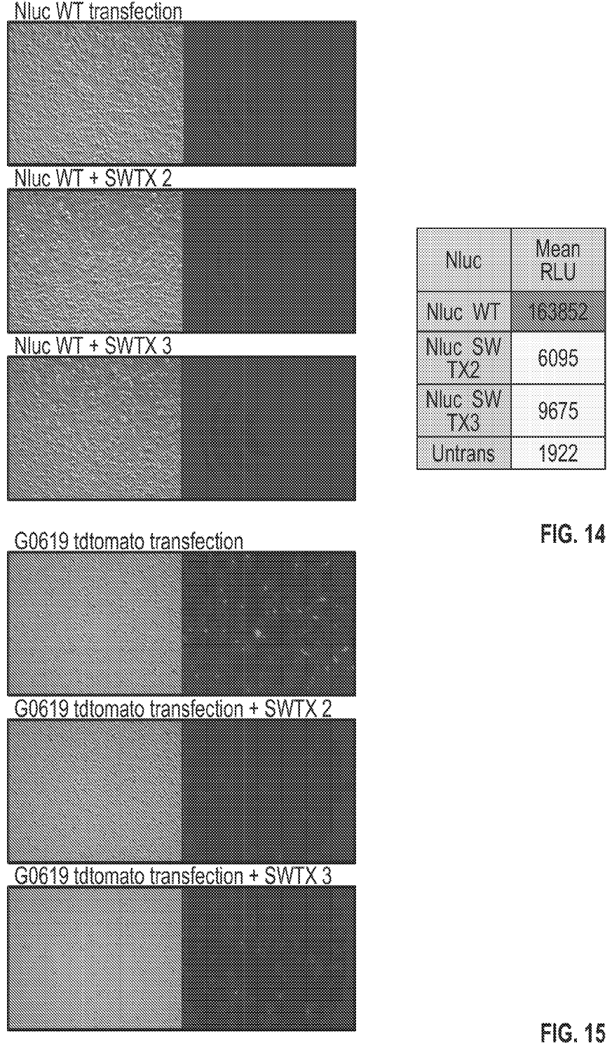

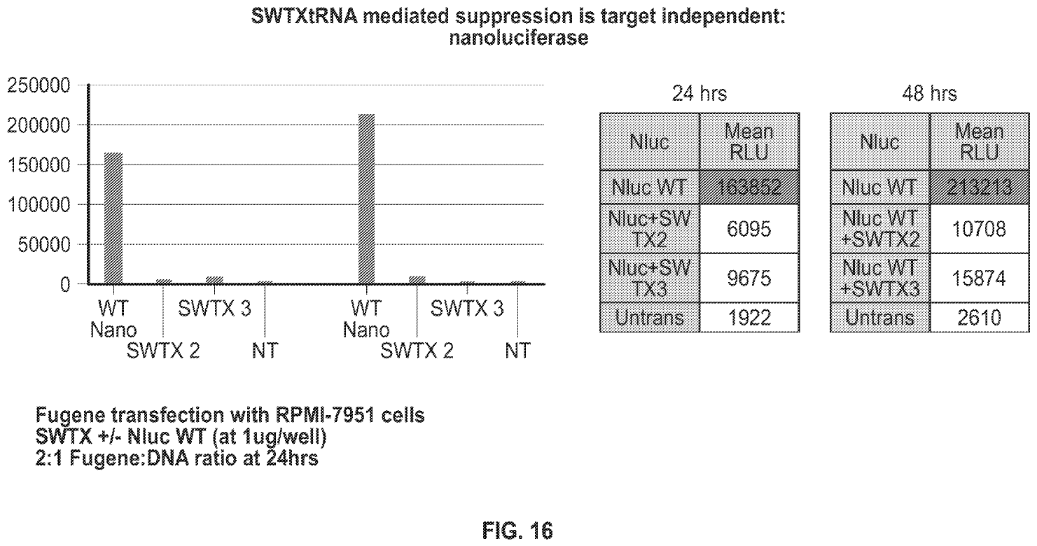

An “all in one” design of the SWTX 51 and 52 tRNAs were used where both tRNA were in the same plasmid payload as the GFP reporter (i.e., a “cis” configuration) to assess the possibility of tRNA cassette promoter interference on GFP expression. SWTX 51 and 52 tRNA were co-expressed in their own plasmid ((called SWTX 2 and SWTX3) separate from the reporter. This is a “trans” conformation. SWTX 2 and SWTX activity was assessed using a melanoma cell line) for tdtomato (a red fluorescent protein) and nanoluciferase expression. SWTX 2 and SWTX3 successfully knocked out tdtomato as well as nanoluc (FIGS. 13 and 14, results at 24 hrs, and FIGS. 15, results at 48 hrs, and 16, results at 24 and 48 hrs).

### Example 5

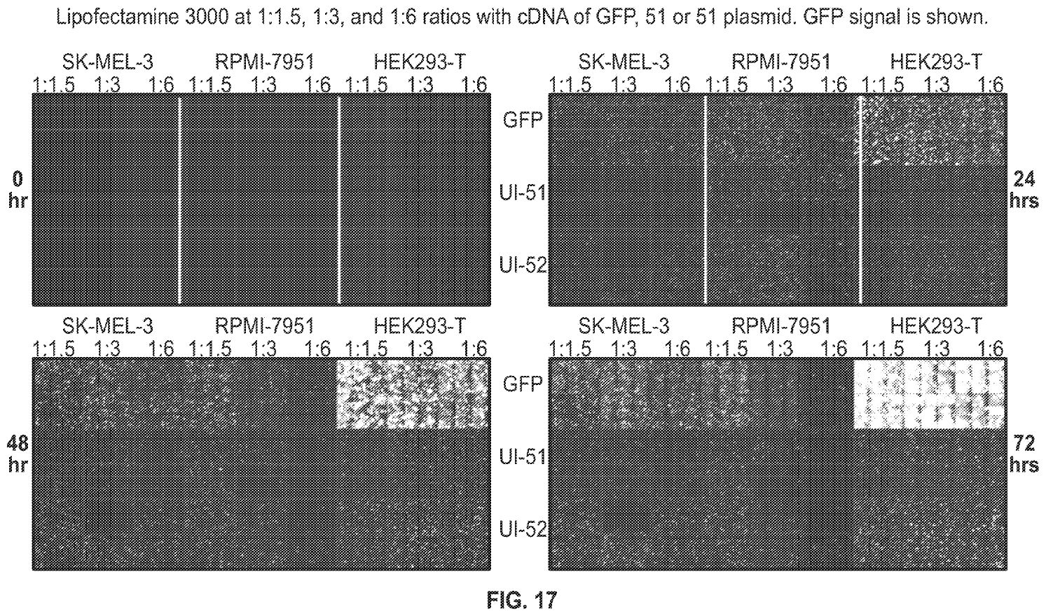

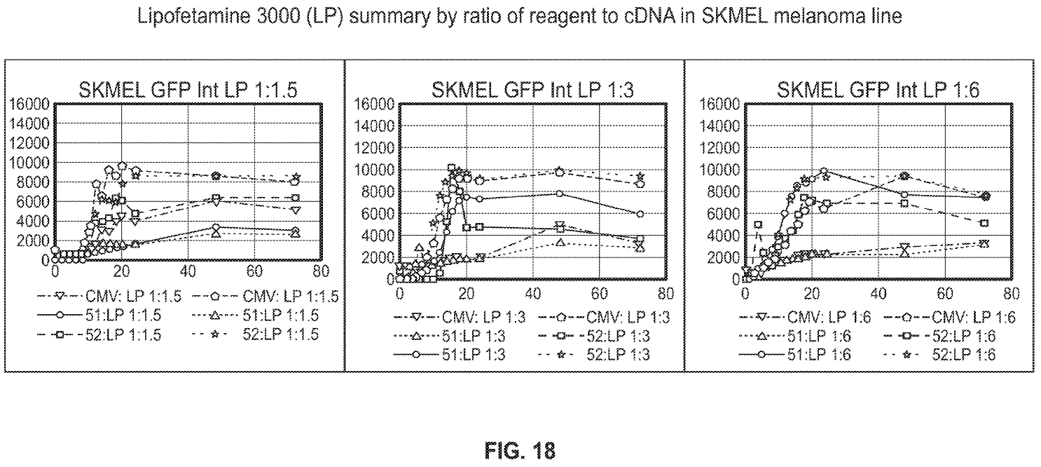

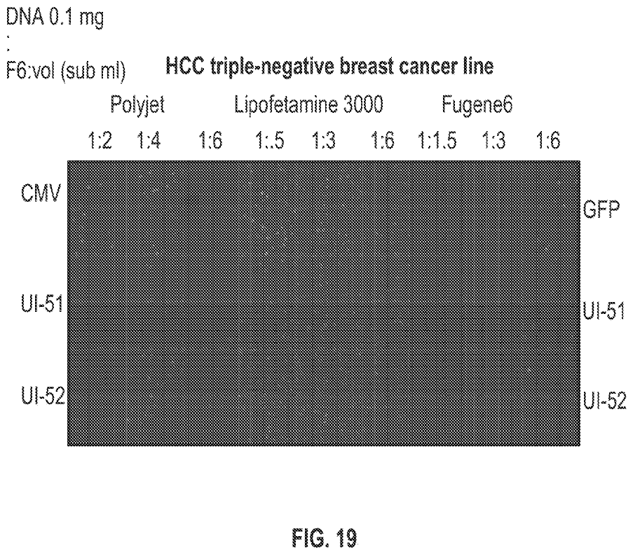

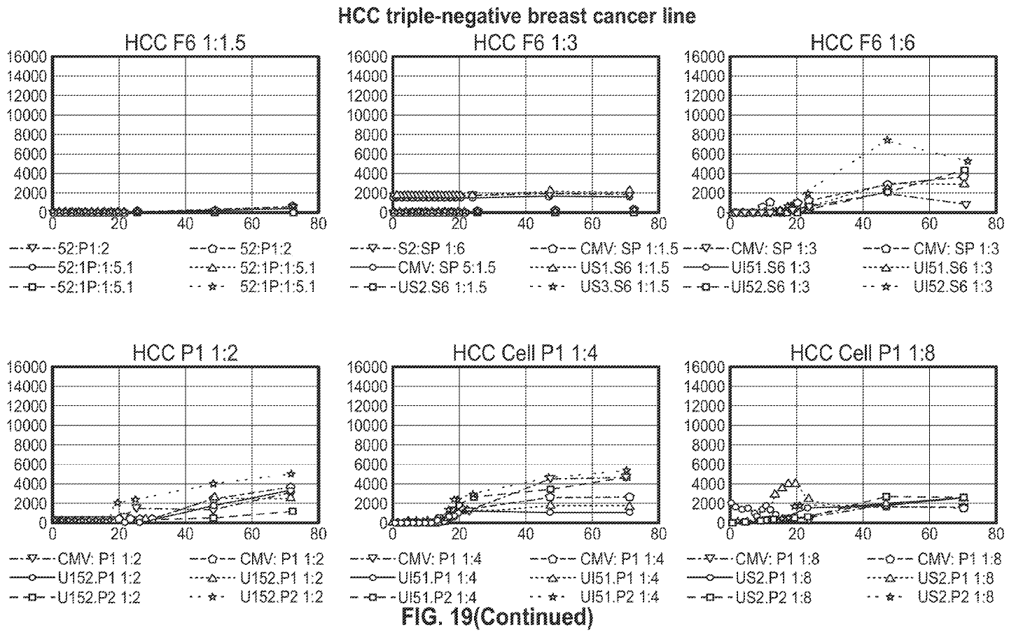

tRNA mediated knock-down of GFP was seen in RPMI-7951 melanoma cells when the tRNA were delivered by lipofectamine and reverse transfection. SWTX 51 and SWTX 52 impaired reporter protein function/expression in a SKMEL melanoma line. Results are shown in a SK Mel 3 melanoma line and HCC triple negative cell line (FIGS. 17-19). For the SK Mel, the RPMI-7951 and HEK comparisons are shown, with WT GFP on the top, followed by SWTX 51 and SWTX 52.

### Example 6

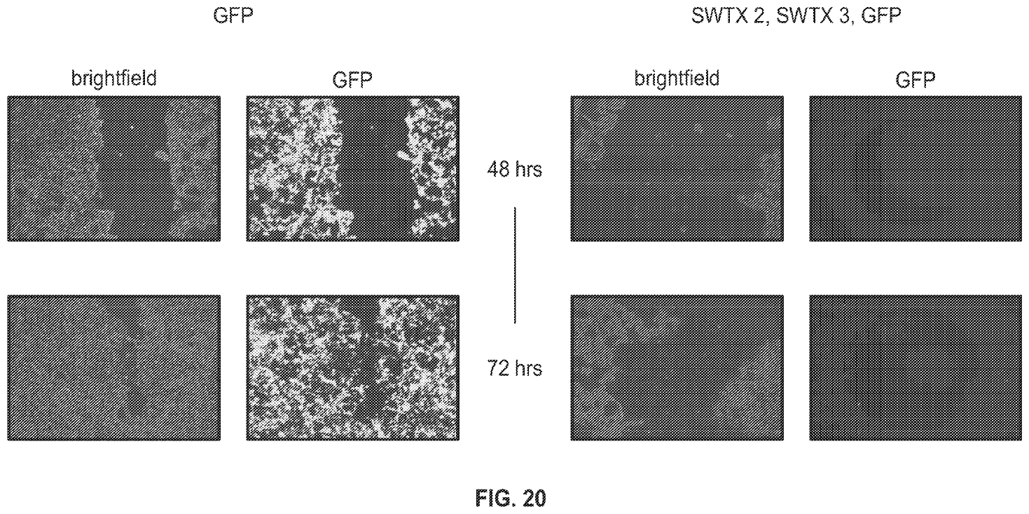

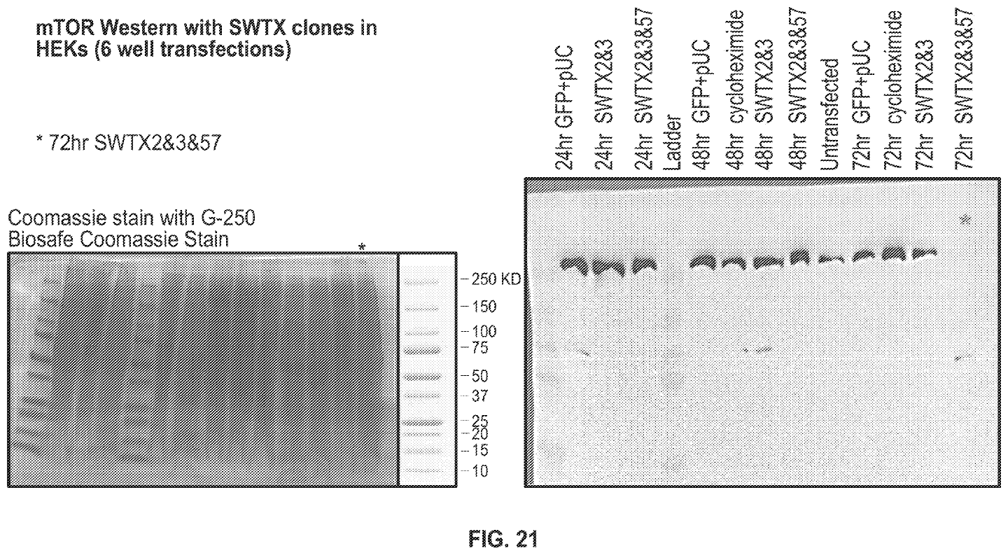

A combination of two constructs: SWTX 2 and SWTX3, the “tRNA alone” versions of SWTX 51 and 52, was used. The data suggest that two tRNA simultaneously attacking the host ribosome can be more potent than a single construct. By 48 hrs (FIG. 20) cell death is seen in the SWTX 2+3 condition but not GFP alone. A scratch assay was performed at 48 hrs to measure the ability of the cells to move back into the cleared area. At 72 hrs, the GFP cells remain healthy, fluorescent and have mostly refilled the clearance made by the scratch. In the SWTX2+3 cells there is wide-spread cell death, no mobility and very little GFP expression.

### Example 7

Using HEK cells and a triple combination of sstRNAs expression of the endogenous metabolic protein, mTOR, a regulator of metabolism was investigated. This protein has greater than 90 proline codons that could be mutated to leucine or isoleucine. The blue stain on the left in FIG. 21 is all protein, to the right in the same figure is the Western against mTOR in a variety of conditions and time points.

A number of combinations of SWTX tRNA were tested, with the triple combination of SWTX Feb. 3, 1957 having the biggest impact at 72 hrs (red asterisk). The level of mTOR is significantly impacted even though the lane is full of protein. The effect is even more pronounced than cyclohexamide which is commonly used to impact the ribosome.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A sense, suppressor transfer RNA (sstRNA) comprising an acceptor stem and an anticodon specific for a noncognate codon.

2. The sstRNA of claim 1, wherein the anticodon is specific for a leucine codon.

3. The sstRNA of claim 2, wherein the acceptor stem is specific for proline.

4. The sstRNA of claim 3, wherein the sstRNA is encoded by a sequence as set forth in SEQ ID NOs: 3 and 4.

5. The sstRNA of claim 1, wherein the anticodon is specific for a proline codon.

6. The sstRNA of claim 5, wherein the acceptor stem is specific for leucine.

7. The sstRNA of claim 6, wherein the sstRNA is encoded by a sequence as set forth in SEQ ID NOs: 1, 2 and 5-13.

8. The sstRNA of claim 5, wherein the acceptor stem is specific for isoleucine.

9. The sstRNA of claim 8, wherein the sstRNA is encoded by a sequence as set forth in SEQ ID NOs: 14 and 15.

10. An oligonucleotide that encodes at least one sstRNA of any one of claims 1 to 9, optionally at least two or three sstRNAs of any one of claims 1 to 9.

11. The oligonucleotide of claim 10, wherein the oligonucleotide has a total length of less than 150 nucleotides or 300 nucleotides.

12. The oligonucleotide of claim 11, wherein the oligonucleotide is DNA, such as cDNA.

13. The oligonucleotide of claim 11, wherein the oligonucleotide is RNA.

14. An expression cassette comprising a promoter and a nucleic acid encoding at least one sstRNA of any one of claims 1 to 9, optionally at least two or three sstRNAs of any one of claims 1 to 9, or the oligonucleotide of any one of claims 10 to 13.

15. The expression cassette of claim 14, further comprising a terminator.

16. An expression cassette comprising a nucleotide sequence as set forth in SEQ ID NOs: 16-26, optionally comprising at least two or three different nucleotide sequences as set forth in SEQ ID NOs: 16-26.

17. A vector comprising the oligonucleotide of any one of claims 10 to 13, or the expression cassette of any one of claims 14-16.

18. The vector of claim 17, wherein the vector is a viral or plasmid vector.

19. A composition comprising:
at least one sstRNA of any one of claims 1 to 9, optionally at least two or three sstRNAs of any one of claims 1 to 9, the oligonucleotide of any one of claims 10 to 13, the expression cassette of any one of claims 14-16, or the vector of claim 17 or 18, and a pharmaceutically acceptable carrier.

20. The composition of claim 19, wherein the pharmaceutically acceptable carrier is a particle, such as a nanoparticle.

21. The composition of claim 20, wherein the particle is a liposome or a lipid nanoparticle.

22. A cell comprising at least one sstRNA of any one of claims 1 to 9, optionally at least two or three sstRNAs of any one of claims 1 to 9, the oligonucleotide of any one of claims 10 to 13, the expression cassette of any one of claims 14-16, the vector of claim 17 or 18, or the composition of any one of claims 19-21.

23. A method of modifying or disrupting protein expression or function, comprising delivering at least one sstRNA of any one of claims 1 to 9, optionally at least two or three sstRNAs of any one of claims 1 to 9, the oligonucleotide of any one of claims 10 to 13, the expression cassette of any one of claims 14-16, the vector of claim 17 or 18, or the composition of any one of claims 19-21 into cells in an amount effective to disrupt protein expression or function in the cells.

24. A method of killing cells, comprising contacting the cells with at least one sstRNA of any one of claims 1 to 9, optionally at least two or three sstRNAs of any one of claims 1 to 9, the oligonucleotide of any one of claims 10 to 13, the expression cassette of any one of claims 14-16, the vector of claim 17 or 18, or the composition of any one of claims 19-21 in an amount effective to kill cells.

25. A method of reducing cell survival, comprising contacting the cells with at least one sstRNA of any one of claims 1 to 9, optionally at least two or three sstRNAs of any one of claims 1 to 9, the oligonucleotide of any one of claims 10 to 13, the expression cassette of any one of claims 14-16, the vector of claim 17 or 18, or the composition of any one of claims 19-21 in an amount effective to reduce the survival of the cells.

26. A method of reducing cell mobility, comprising contacting the cells with at least one sstRNA of any one of claims 1 to 9, optionally at least two or three sstRNAs of any one of claims 1 to 9, the oligonucleotide of any one of claims 10 to 13, the expression cassette of any one of claims 14-16, the vector of claim 17 or 18, or the composition of any one of claims 19-21 in an amount effective to reduce the mobility of the cells.

27. A method of activating immune cells, comprising contacting cells with at least one sstRNA of any one of claims 1 to 9, optionally at least two or three sstRNAs of any one of claims 1 to 9, the oligonucleotide of any one of claims 10 to 13, the expression cassette of any one of claims 14-16, the vector of claim 17 or 18, or the composition of any one of claims 19-21 in an amount effective to activate immune cells.

28. The method of any one of claims 23-27, wherein the cells are in vitro.

29. The method of any one of claims 23-27, wherein the cells are in vivo.

30. A method of treating a subject with a hyperproliferative disease or disorder, comprising administering to the subject at least one sstRNA of any one of claims 1 to 9, optionally at least two or three sstRNAs of any one of claims 1 to 9, the oligonucleotide of any one of claims 10 to 13, the expression cassette of any one of claims 14-16, the vector of claim 17 or 18, or the composition of any one of claims 19-21 in an amount effective to treat the hyperproliferative disease or disorder.

31. The method of claim 30, wherein the hyperproliferative disease or disorder is cancer.

32. The method of claim 31, wherein the cancer is melanoma or breast cancer, such as triple negative breast cancer.

33. The method of any one of claims 23-32, wherein when there is at least two or three sstRNAs the at least two or three sstRNAs are in the same oligonucleotide, expression cassette, vector or composition.

34. The method of any one of claims 23-32, wherein when there is at least two or three sstRNAs the at least two or three sstRNAs are in at least two or three different oligonucleotides, expression cassettes, vectors or compositions.
